(19) United States
(12) Patent Application Publication
(10) Pub. No.: US 2009/0131833 A1
(43) Pub. Date: May 21, 2009

(54) FETAL HEAD MONITORING DEVICE

(75) Inventor: Andrew Weeks, Liverpool (GB)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: MITSUBISHI ELECTRIC CORPORATION

(21) Appl. No.: 12/282,299

(22) PCT Filed: Mar. 9, 2007
(86) PCT No.: PCT/GB07/00837

§ 371 Date: Jan. 8, 2009

(30) Foreign Application Priority Data
Jan. 12, 1988 (JP) 63-005650
Sep. 13, 1989 (JP) 01-237455
Sep. 3, 1996 (JP) 08-233038
Jan. 16, 2001 (JP) 2001-007785
May 20, 2003 (JP) 2003-142629

(51) Int. Cl. A61B 17/42; A61B 5/11

(52) U.S. Cl. 600/588; 606/125

(57) ABSTRACT

A monitoring device (1) for internal vaginal examinations during labour to assess descent of the fetal head through the maternal pelvis. The device (1) comprises first (2) and second (3) sections mounted together so as to be co-axially extendable and retractable relative to each other to vary the distance between opposite free ends (2a) and (3b) of the device (1) and being adapted to be releasably restrained together at a plurality of selectable co-axial positions. The second section (3) has a reference mark (7) for use in setting the device for a patient to be examined so that the distance between the free end (2a) of the first section (2) and the reference mark (7) corresponds to the distance in the patient between the posterior fourchette and the plane containing the ischial spines.

The present invention relates to a fetal head monitoring device for use in internal vaginal examinations during labour to assess descent of the fetal head.

Approximately 10-15% of births are assisted with the use of forceps or a ventouse. Such assistance may be required for example where the mother has been administered an epidural and due to lack of “sensation” is unable to “push” to the degree necessary for an unassisted birth.

The use of forceps, ventouse or other assist device does however carry the risk of damage to the fetal head and maternal soft tissues, particularly since the location of the head within the pelvis may not be known with sufficient accuracy for the safe use of such assist devices. The use of assist devices when the head is high in the pelvis is associated with higher risks of complications for both mother and baby. There are two techniques for assessing descent of the fetal head. The first is by examining the maternal abdomen to palpate the amount of fetal head remaining above the pelvic brim. Whilst a useful test, this technique cannot be used in a woman who is overweight or unable to relax her abdominal muscles (i.e. if she is in pain or distressed). Both of these situations (pain and distress) are common in the late stages of labours making this technique often unusable. The second technique relies on a medical practitioner inserting their fingers into the vagina of the patient to feel the sutures on the fetal head and then assessing the head position with respect to the ischial spines, the two bony projections lying in a plane within the pelvis to either side of the vagina. This position (at the ischial spines) is known as “Station 0” and positions further down the vagina are referred to as “+1”, “+2”, etc. It is however extremely difficult to assess accurately the exact position of the baby's head during such examination with consequential uncertainty as to whether an assist device such as forceps or a ventouse is safe to use.

It is therefore an object of the present invention to obviate or mitigate the aforementioned disadvantages.

According a first aspect of the present invention there is provided a monitoring device for internal vaginal examinations during labour to assess descent of the fetal head through the maternal pelvis, the device comprising first and second elongate sections mounted together so as to be co-axially extendable and retractable relative to each other to vary the distance between opposite free ends of the device and being adapted to be releasably restrained together at a plurality of selectable co-axial positions, said second section having a reference mark for use in setting the device for a patient to be examined so that the distance between the free end of the first section and the reference mark corresponds to the distance in the patient between the posterior fourchette and the plane containing the ischial spines.

According a second aspect of the present invention there is a provided a method of assessing descent of the fetal head during labour of a patient, the method comprising the steps of:

(i) setting a device as defined in the previous paragraph so that the distance between the free end of the first section and the reference marking on the second section corresponds to the distance, in the patient, between the posterior fourchette and the plane containing the ischial spines; and

(ii) periodically assessing the position of the fetal head by locating the free end of the first section in contact with a leading portion of the descending fetal head and assessing the position of the fetal head in relation to displacement of the reference marking relative to the posterior fourchette.

The device of the invention relies on being set (for a particular female patient giving birth) such that the distance between the free end of the first section and the reference mark on the second section corresponds to the distance (in that patient) between the posterior fourchette and the plane containing the ischial spines. The manner in which this can conveniently be achieved is described more fully below.

Once the device has been set it may be used for assessing the position of the descending fetal head in a number of ways. For all assessments, the free end of the first section is positioned within the vagina so as to be in contact with a leading portion of the descending fetal head. Given that the head has usually moved beyond of the plane of the ischial spines, the reference marking on the second section will be located outwardly of the posterior fourchette by a distance corresponding to that by which the leading portion of the head has moved past the ischial spines. Alternatively or additionally the distance from the leading part of the descending fetal head to the posterior fourchette may be determined as the difference between the distance originally “set” on the device (i.e. the distance between the free end of the first section and the reference marking on the second section) and the extent to which the reference marking has been displaced outwardly beyond the posterior fourchette.

Positional assessments of the descending fetal head obtained using the device of the invention allow accurate use of birth assist devices such as forceps or a ventouse thus potentially reducing the possibility of damage being inflicted by such devices. It may also be used repeatedly through the course of labour in order to assess the normal descent of the fetal head during labour and allowing early intervention if deviations from this norm occur.

Conveniently the second section is provided with a measurement scale (e.g. in centimeters). The reference mark may be the “zero” on such a measurement scale and the scale may increase positively (e.g. +1, +2 etc) going in a direction along the second section in a direction towards the free end of the first section. The scale may extend “negatively” in the opposite direction along the second section.

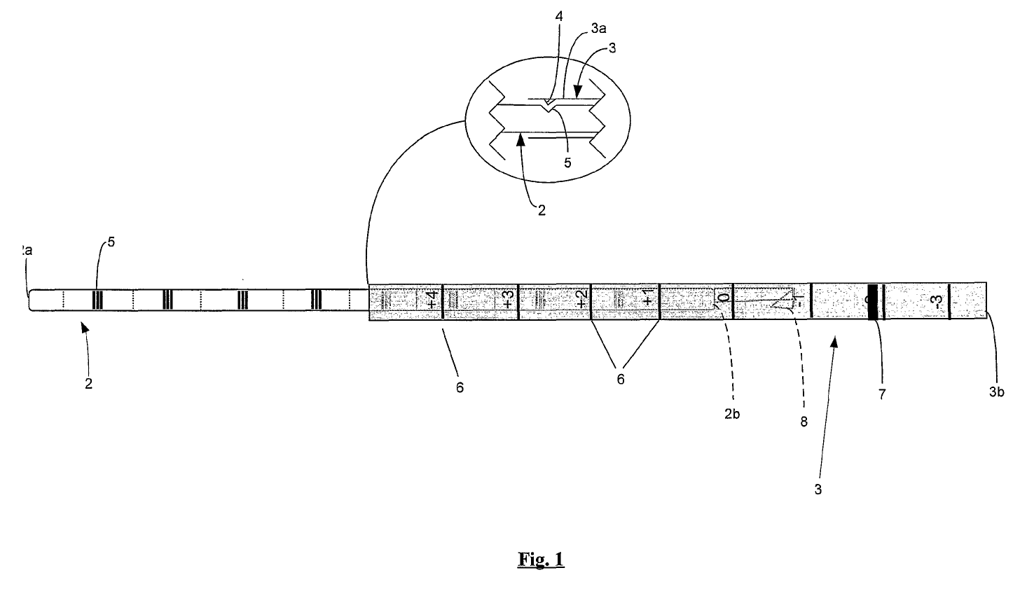

Conveniently, the second section is provided with a “correction marking” which is about 1.0-2.5 cm from the reference marking (e.g. “zero” on the measurement scale) going in a direction away from the free end of the first section. As will be understood from the description given below in relation to FIGS. 1-3, such a correction marking enables the device to be “set” to the required length by locating the free end of the first section on an ischial spine and the correction marking on the posterior fourchette. Preferably the “correction marking” is about 1.7-2.1 cm from the reference marking and ideally about 1.9 cm therefrom.

Preferably the first section is also provided with regular measurement markings to be read in conjunction with those on the second section. The presence of the markings provides a continuous scale along the length of the first and second sections, thereby allowing the total distance from the free end of the first section to the reference mark to be read (e.g. in centimetres) irrespective of how far the first section is extended relative to the second section. Thus once the device has been “set”, the distance between the posterior fourchette and the plane of the ischial spines can readily be determined by inspection of the device. This has advantages in determining the distance that the leading part of the fetal head needs to travel to reach the posterior fourchette. More particularly (as discussed above) descent of the fetal head past ischial spines will mean that, when the device is used, the reference (zero) marking on the second section will be located outwardly of the posterior fourchette. The distance by which the marking is so displaced will give the position of the head relative to the posterior fourchette.

In the device of the invention, the first and second sections are adapted to be releasably restrained together at a plurality of selectable co-axial positions. This ensures that the device may be set to any length as required and the first and second sections cannot be displaced relative to each other (thereby offsetting the original measurement) at least without sufficient axial force being applied to displace the first and second sections relative to each other. Any convenient form of complementary detent formations on the first and second sections may be used to provide this functionality.

Preferably the second section is a tubular sheath within which at least a part of the first section is slideable. Alternatively the sheath may be of C-shaped cross-section. Conveniently, the first section has a plurality of axially spaced, transverse detent formations (e.g. grooves or ridges) which cooperate with a tooth or the like provided on the sheath whereby the first section and the sheath may be retained at selected co-axial positions relative to each other. Such transverse detent formations on the first section will preferably be regularly spaced and may additionally serve as the aforementioned measurement markings for this section. Alternatively or additionally, the two terminal transverse detent formulations may be configured such that the first section is prevented from sliding out of the sheath.

The device of the invention may be provided with a sharp (e.g. needle-like) formation for use rupturing the amniotic membrane. Conveniently such a formation is a so-called “amniotomy hook”. In a particularly preferred embodiment of the invention, the second section is a sheath and the formation for rupturing the anmiotic membranes is provided on the first section such that said formation can be withdrawn within the sheath and used before or after the position of the fetal head has been assessed.

Alternatively or additionally, the free end of the inner section may be provided with a fetal heartbeat monitor capable of sending a signal to a receiving device (incorporating a digital display of heartbeat rate.

Devices in accordance with the invention may conveniently be made of plastics material.

Preferably the device is used in a sterile condition. Sterilisation may be achieved by any conventional means. The device may be supplied within a bacteria impervious pack.

The invention will be further described by way of example only with reference to the accompanying drawings, in which:

FIG. 1 illustrates one embodiment of monitoring device (to an enlarged scale) in accordance with the invention;

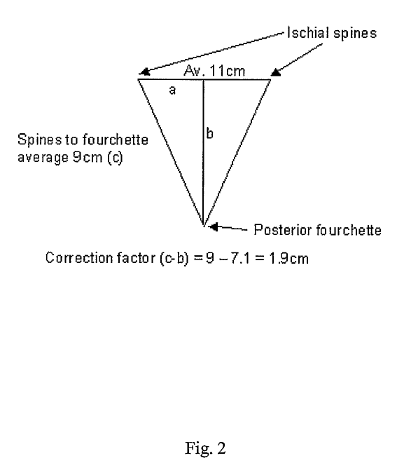

FIG. 2 illustrate the manner in which a correction factor is calculated for the device of FIG. 1; and

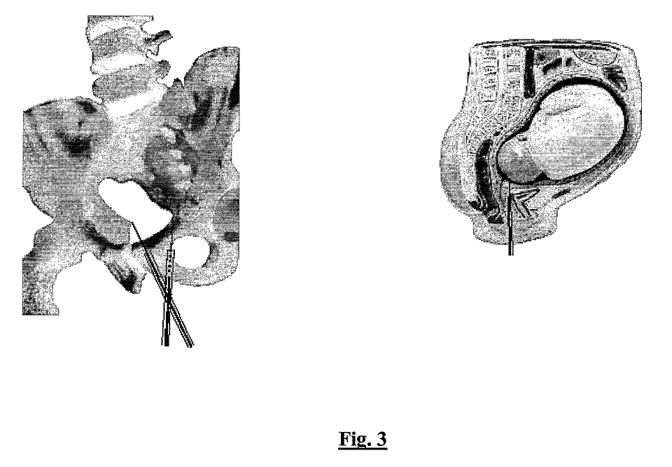

FIG. 3 illustrates use of the device of FIG. 1.

As shown in FIG. 1, a fetal head monitoring device 1 (which is conveniently fabricated from synthetic plastics material) comprises an elongate first section in the form of a generally round probe 2 (depicted as having ends 2a and 2b) and an elongate second section formed as a sheath 3 (depicted as having ends 3a and 3b) which accommodates probe 2. Typically probe 2 will have a length of 21 cm and sheath 3 will have a length of 18 cm. Sheath 3 may, for example, be tubular or of C-shaped cross-section, if the latter, the probe 2 may be “clipped” into the sheath 3.

Probe 2 is slidable within sheath 3 so that the overall length of the device as measured between the free end 2a of probe 2 and the (opposite) free end 3b of sheath 3 may be varied.

Although not illustrated in the drawings, the ends 3a and 3b of sheath 3 and end 2a of probe 2 are preferably rounded (“rolled”) to prevent catching of the ends on tissue.

Sheath 3 may be conveniently provided at its end 3a with an integral tooth 4 which, as shown in the insert to FIG. 1, projects a short distance into the bore of sheath 3. Additionally, probe 2 is formed at 5 nm intervals with axially spaced notches 5 (which also serve as measurement markings) each extending transversely across probe 2. The tooth 4 and notches 5 are so formed that the tooth 4 can releasably engage within any one notch 5 to locate probe 2 relative to sheath 3 until sufficient force is applied along the length of the device to cause tooth 4 to disengage from the notch 5 and allow the device to be extended or retracted as required. The terminal notches 5 (i.e. those closest to the ends 2a and 2b of the probe 2) are preferably widened so as to provide a greater degree of “engagement force” with the tooth 4 than is the case for the other notches 5 and thereby prevent the outer sheath 3 sliding off the inner probe 2.

A measuring scale 6 is provided along sheath 3. Conveniently scale 6 is graduated in centimeters although other units and/or sub-divisions could be used. More specifically, scale 6 has a “zero” value 5 cm to the right (as viewed in FIG. 1) of the end 3a of sheath 3 and the scale goes positive (i.e. +1 cm, +2 cm etc) in a direction towards end 3a and negative (−1 cm, −2 cm etc) in the other direction. Although not illustrated in the drawing, the “negative markings” may (for an 18 cm long sheath 3) go down to minus 13 cm.

Also marked on the scale 6 is a correction marker 7 which is represented in the illustrated embodiment as being positioned at −1.9 cm on the scale, i.e. 1.9 cm to the right (as viewed in FIG. 1) of the zero value. Correction marker 7 is shown as a dark bar of greater thickness than any of the other markings and is therefore visually distinct. Alternatively or additionally, correction marker 7 could be of a contrasting colour as compared to the other markings.

A further feature of the illustrated device is an anmiotomy hook 8 (providing a sharp needle-like point) provided at the end 2b of probe 2.

It is envisaged that device 1 will be disposable (i.e. “use once”) and will be supplied in a package in a condition in which the amnihook 8 is exposed by 2-3 cm beyond the end 3b of sheath 3.

Additionally it is envisaged that the device 1 will be supplied in a sterile condition.

A full description of the manner in which the device is used will be given below but it is convenient at this point to explain (with reference to FIG. 2) the purpose and position of correction marker 7.

In use for monitoring descent of the fetal head, the device 1 is intended to be set so that the distance between the free end 2a of probe 2 and the “zero” marker on scale 6 corresponds to the distance (in the particular female patient concerned) from the plane containing the ischial spines to the posterior fourchette. This distance can vary from one female patient to another and the device 1 therefore needs to be set for the patient concerned. This “setting” procedure is intended to be effected with the device 1 positioned partially in the vagina with end 2a first. In this regard, it would be relatively easy to locate the “zero” marker of scale 6 on the posterior fourchette but it would be extremely difficult to know with any certainty that end 2a was in the plane of the ischial spines.

It is however relatively easy to locate end 2a of probe 2 on a ischial spine but the distance between the end 2a and the “zero” marker (assuming the latter was positioned on the posterior fourchette) is then not the actual distance required. This is where correction marker 7 comes in.

Consider now FIG. 2. This shows an isosceles triangle for which the two upper (as viewed in FIG. 2) apices are intended to represent the positions of the ischial spines and the lower apex represents the position of the posterior fourchette. It will be appreciated that the upper side of the triangle represents the plane containing the ischial spines.

Dimensions a, b and c are shown on the triangle and have the following meanings.
A=half the distance between the ischial spines.
B=the distance between the posterior fourchette and the plane containing the ischial spines.
C=the distance between the ischial spine and the posterior fourchette.

The following average values may be assumed:

2a=11 cm (i.e. a=50.5 cm)

c=9 cm

b=7.1 cm (by Pythagoras)

(c-b)=1.9 cm

It will be appreciated that with the end 2a of probe 2 on an ischial spine and the correction marker 7 on the posterior fourchette the distance between end 2a and marker 7 is effectively (b+1.9). Therefore the distance between end 2a and the “zero” marker on scale 6 represents b, i.e. the distance between the posterior fourchette and the plane containing the ischial spines. The value of 1.9 is therefore a “correction factor” which considerably facilitates “setting” of the device 1.

It should be appreciated at this point that the distances a, b and c may vary from female-to-female but the correction factor of 1.9 cm is presently regarded as sufficiently accurate for use of the device on all females. It is of course possible to calculate possible upper and lower limits for the value of (c-b) by considering different female anatomies. Thus, at one extreme, a small corpulent female may be such that a=4.5 cm (2a=9.0 cm) and c=15 cm thus giving a value of b=14.3 cm and (c-b)=0.7 cm. At the other extreme, very large boned, lean female may be such that a=7 cm and c=10 cm so that b=7.1 cm and (c-b)=2.9 cm. Thus, in theory, extremes of correction factors of 0.7 cm and 2.9 cm may be envisaged but these extreme values are unlikely to be encountered given that for most women the measurements of a and c will increase together. In practice, the value of (c-b) is most likely to lie in the range of 1 to 2.5 cm with 1.9 cm being a good “working value”. It should however be understood that it would be possible to produce a range of devices in accordance with the invention with different values of the correction factor to cater for different anatomies. A further possibility is a single device marked with two or more correction factors, the appropriate one being selected for use in a particular application.

A fuller description of the use of the device 1 will now be given.

Initially, it will be a matter for the medical personnel supervising the birth to decide whether or not the amnihook should be used for rupturing the amniotic membranes.

Probe 2 is now extended outwardly of sheath 3 (so that amnihook 8 is located within the latter) and the device “set” in the manner indicated above, i.e. locating the end 2a of probe 2 of an ischial spine and extending/retracting probe 2 and sheath 3 relative to each other so that correction marker 7 is positioned on the posterior fourchette (see also FIG. 3). It should however be ensured that the tooth 4 on the sheath 3 locates in a notch 5 (thereby ensuring that the length of the device becomes “fixed”) so some slight extension or retraction of the probe 2 relative to sheath 3 may be required to ensue this condition applies.

Once the device has been withdrawn from the vagina, the actual distance in the patent concerned between the plane of the ischial spines and the posterior fourchette may easily be determined by inspection as the distance from “zero” mark on scale 6 to end 2a ofprobe 2. More particularly this is 5 cm (i.e. the distance from the “zero” mark to end 3a of sheath 3) plus the extent to which end 2a of probe 2 projects beyond sheath 3. This latter dimension may be readily determined from the markings (provided by the notches 5) on probe 2.

During labour, the device 1 (set to length as described above) may be used for internal vaginal examinations by positioning end 2a of probe 2 in contact with the leading part of the fetal head and the sheath 3 on the posterior fourchette.

Given that the leading part of the fetal head has passed through and below the ischial spines then the scale 6 may be used to determine how far the head has passed beyond the ischial spines. Thus, say, the +2 marking is at the posterior fourchette then it is known that the leading part of the fetal head is 2 cm past the ischial spines. Additionally it will also be known that the leading part of the fetal head is (b-2) cm above the posterior fourchette.

The progress of the fetal head may be monitored by making periodic measurements of this type.

Should it be necessary to use forceps or a ventouse to assist the birth then a very good indication of the position of the leading part of the head below the plane of the ischial spines at the time the assistance is required can be obtained. This allows for accurate assessment of the likely success or failure of the procedure. For those with a high chance of failure, a caesarean section may be performed in preference, preventing the complications caused by a failed delivery with forceps or ventouse.

It will be appreciated that it is possible to make a number of modifications to the illustrated device. Thus, for example, each 0.5 cm section of the probe 2 (i.e. between adjacent markings thereon) may carry a different letter of the alphabet. The device may be “set” for an internal vaginal examination as described above. There will now be a particular letter on probe 2 positioned immediately to the left (as viewed in FIG. 1) of end 3a of sheath 3. This letter may be recorded in the patient's records so that on further occasions (e.g. further examinations in the same labour or in future labours) where a device 1 is to be used for that patient the device may simplybe adjusted (externally of the vagina) to the required length on the basis of the information in the patient's records without need for a detailed internal examination.

1. A monitoring device for internal vaginal examinations during labour to assess descent of the fetal head through the maternal pelvis, the device comprising first and second elongate sections mounted together so as to be co-axially extendable and retractable relative to each other to vary the distance between opposite free ends of the device and being adapted to be releasably restrained together at a plurality of selectable co-axial positions, said second section having a reference mark for use in setting the device for a patient to be examined so that the distance between the free end of the first section and the reference mark corresponds to the distance in the patient between the posterior fourchette and the plane containing the ischial spines.

2. A device as claimed in claim 1 wherein the second section is provided with a measurement scale.

3. A device as claimed in claim 2 wherein the measurement scale is in centimetres.

4. A device as claimed in claim 2 wherein the reference mark is “zero” on the measurement scale.

5. A device as claimed in claim 4 wherein the scale increases positively going in a direction along the second section in a direction towards the free end of the first section.

6. A device as claimed in claim 1 wherein the second section is provided with a “correction marking” which is about 1.0-2.5 cm on the side of the reference marking going in a direction away from the free end of the first section.

7. A device as claimed in claim 6 wherein the correction marking is about 1.9 cm on the side of the reference marking going in a direction away from the free end of the first section.

8. A device as claimed in claim 1 wherein the first section is provided with measurement markings.

9. A device as claimed in claim 1 wherein the first and second sections are provided with complementary detent formations.

10. A device as claimed in claim 1 wherein the second section is a tubular sheath within which at least a part of the first section is slideable.

11. A device as claimed in claim 1 wherein the second section is a C-section sheath within at least a part of the first section is slideable.

12. A device as claimed in claim 10 wherein the first section has a plurality of axially spaced, transverse detent formations which cooperate with a tooth or the like provided on the sheath whereby the first and second sections may be retained at selected co-axial positions relative to each other.

13. A device as claimed in claim 1 made of plastics material.

14. A device as claimed in claim 1 provided with a sharp formation for use in rupturing the amniotic membrane.

15. A device as claimed in claim 14 wherein said formation is an amniotomy hook.

16. A device as claimed in claim 14 wherein the second section is a sheath and the formation for rupturing the amniotic membranes is provided on the first section such that said formation can be withdrawn within the sheath.

17. A device as claimed in claim 1 provided with a fetal heartbeat monitor at the free end of the first section.

18. A device as claimed in claim 1 which is sterile.

19. A method of assessing descent of the fetal head during birth labour of a patient, the method comprising the steps of:
(i) setting a device as claimed in claim 1 so that the distance between the free end of the first section and the reference marking on the second section corresponds to the distance, in the patient, between the posterior fourchette and the plane containing the ischial spines; and
(ii) periodically assessing the position of the fetal head by locating the free end of the first section in contact with a leading portion of the descending fetal head and assessing the position of the fetal head in relation to displacement of the reference marking relative to the posterior fourchette.
